(19) United States
(12) Patent Application Publication
(10) Pub. No.: US 2013/0023469 A1
(43) Pub. Date: Jan. 24, 2013

(54) LEPTIN FOR USE IN INCREASING LIVER REGENERATION

(71) Applicants: Eli Pikarsky, Jerusalem (IL); Yehudit Bergman, Jerusalem (IL); Neri Laufer, Jerusalem (IL); Yuval Gielchinsky, Jerusalem (IL); Efraim Weitman, Givat Shmuel (IL)

(72) Inventors: Eli Pikarsky, Jerusalem (IL); Yehudit Bergman, Jerusalem (IL); Neri Laufer, Jerusalem (IL); Yuval Gielchinsky, Jerusalem (IL); Efraim Weitman, Givat Shmuel (IL)

(73) Assignees: Hadasit Medical Research Services & Development Ltd., Jerusalem (IL); Yissum Research Development Company of the Hebrew University of Jerusalem Ltd., Jerusalem (IL)

(21) Appl. No.: 13/497,661

(22) PCT Filed: Sep. 21, 2010
(86) PCT No.: PCT/IL10/00790

§ 371 Date: Oct. 9, 2012

Related U.S. Application Data
(60) Provisional application No. 61/244,548, filed on Sep. 22, 2009.

(51) Int. Cl. A61K 38/22; A61P 1/16

(52) U.S. Cl. 514/5.8

(57) ABSTRACT

Activators of the AKT/mTOR pathway, in particular leptin, are useful in methods and compositions for increasing regeneration of liver, increasing liver mass or improving liver function, or a combination thereof.

### TECHNICAL FIELD

The present invention relates to the field of increasing liver regeneration, in particular in aging populations, by the use of AKT/mTORc1 pathway activators.

### BACKGROUND

In aging organisms, tissue regenerative capacity declines and healing in response to injury is delayed. This effect—which is observed in liver, skin, bone, hematopoietic system, blood vessels, nerve, and muscle—is attributable to the altered functions of many biological processes. These include changes in growth factors or in extracellular matrix components, accumulation of DNA damage, increased presence of intracellular oxygen-reactive species, and decline in responsiveness of progenitor cells. Liver regeneration, a process that rapidly compensates for the acute loss of liver parenchyma in patients with liver tumors or fulminant hepatitis (Michalopoulos 2007), is widely used as a model of tissue regeneration and surgical stress, a major problem in the geriatric population. Studies have shown that, in old mice, the liver regenerates significantly more slowly than in young mice. This effect is already seen in 1-yr-old rats and mice (FIG. 1). This decline has therapeutic relevance, as surgical resection is often the best option in patients with primary or secondary hepatic malignancies. However, given the considerable increase (by 2% per year) in the odds ratio for mortality in the aged population, devising ways to improve liver regeneration in older patients is of paramount clinical importance.

### SUMMARY OF INVENTION

The present invention relates, in one aspect, to an activator of the AKT/mTOR pathway, in particular leptin, for use in increasing regeneration of liver, increasing liver mass or improving liver function, or a combination thereof, and in related aspects to methods for improving liver regeneration, increasing liver mass or both in a subject in need of such treatment, the method comprising administering to said subject an activator of the AKT/mTOR pathway.

In another aspect the present invention provides leptin for use in increasing regeneration of liver, increasing liver mass, or improving liver function, or a combination thereof.

In an additional aspect, the present invention provides a pharmaceutical composition for improving liver regeneration, increasing liver mass, or improving liver function, or a combination thereof, said pharmaceutical composition comprising an activator of the present invention and a pharmaceutically acceptable carrier.

In still another aspect, the present invention provides use of an activator of the present invention for the preparation of a medicament for improving liver regeneration increasing liver mass, or improving liver function, or a combination thereof.

### BRIEF DESCRIPTION OF DRAWINGS

FIG. 1 shows that the capacity for liver regeneration declines with age. 3-month-old (triangles), 10-12-month-old (diamonds) and >18-month-old (crosses) nonpregnant female mice were subjected to ⅔ partial hepatectomy. In each mouse, liver volume was determined by MRI on the indicated days and recorded as a percentage of the liver volume before partial hepatectomy (mean±s.e.m.). Note that while the age effect may seem to be transient, it results in considerable mortality.

FIGS. 2A-C show that pregnancy improves liver regeneration in aged mice. (A) Representative serial MRI images of individual aged mice on the indicated days after ⅔ partial hepatectomy. Hatched lines denote the liver contours. Scale bar on picture represents 1 cm. (B) Photographs of representative livers of aged mice removed 2 days after surgery. (C) For each mouse, liver volume on days 0, 1, 2, and 5 was determined by MRI and recorded as a percentage of the liver volume prior to partial hepatectomy (mean±s.e.m). *P values were calculated for aged pregnant mice (n=5) relative to aged nonpregnant mice (n=5), using Student's t test. Diamonds, aged non-pregnant; Squares, aged pregnant; Triangles, young non-pregnant; Crosses, young pregnant. Aged, 10-12 months-old; Young, 3 months old.

FIGS. 3A-B depict improved recovery of liver function in pregnant mice after partial hepatectomy. 10-12 month old pregnant and nonpregnant mice were subjected to partial hepatectomy. (A) Blood was collected 24 h after partial hepatectomy and prothrombin time was measured (mean±s.e.m., n=3, P<0.05, Kruskal-Wallis test). Prothrombin time values in nonhepatectomized controls ranged between 11.0 and 12.3 seconds (dashed line). (B) Locomotor activity of mice in the indicated groups, 1 day after partial hepatectomy, was monitored by an open-field recorder as detailed in the materials and methods section (n≧4; P<0.0001, Mann-Whitney test). First (black) bar from the left (diamonds), 10-12 months-old non-pregnant; 2^{nd} bar from the left (squares), 10-12 months-old pregnant; 3^{rd} bar from the left (asterisks), 18-24 months old non-pregnant; 4^{th} bar from the left (triangles), 18-24 months old non-pregnant+bpV(phen).

FIGS. 4A-D show that liver regeneration in pregnancy proceeds via the hypertrophy module. (A) Percentage of BrdU-positive cells on the indicated days after ⅔ partial hepatectomy in aged mice. Nonpregnant (squares) and pregnant (asterisks) mice were injected with 5-bromo-2-deoxyuridine (BrdU) at the indicated time points after partial hepatectomy. BrdU incorporation into hepatocytes was assayed using immunohistochemistry. Each data point represents a single mouse of the indicated groups. P<0.05, nonparametric linear regression (B) Immunohistochemical staining for BrdU in aged mice. To rule out the possibility that a specific time point at which hepatocytes in pregnant mice enter the S-phase was missed, BrdU was administered in the drinking water from the time of partial hepatectomy until 4 days after the surgical procedure, when the mice were sacrificed. Scale bars represent 100 μm. P=0.002, Student's t test. In the left panel, 83±7% was labeled; In the right panel, 6±4% was labeled; (C) Representative E-cadherin-stained images demonstrating changes in cell size. Scale bars represent 20 μm. In the upper left panel, cell size was 307±29μ²; In the upper right panel, cell size was 347±35μ²; In the lower left panel, cell size was 424±13μ²; and in the lower right panel, cell size was 703±28μ²; (D1-4). Cell-size distribution in the livers of aged nonpregnant (asterisks) and pregnant (circles) mice on the indicated days after surgery. Each data point represents two or three mice. For each mouse four fields were counted, harboring a total of at least 100 cells. (E) Average cell size in livers of aged nonpregnant (diamonds) and pregnant (squares) mice on the indicated days after surgery (mean±s.e.m.). P=0.0001, Mann-Whitney test.

FIGS. 5A-B show that BrdU incorporation in the bowel is not affected by pregnancy. Representative immunohystochemical staining for BrdU of small bowels from non-pregnant (A) and pregnant (B) mice. BrdU was administered continuously in the drinking water from the time of partial hepatectomy until sacrifice four days later.

FIGS. 6A-C show that there is hypertrophy of hepatocytes in hepatectomized pregnant mice. (A) Aged pregnant and nonpregnant mice were subjected to partial hepatectomy. Four days after surgery mice were re-anesthetized and single-cell suspensions of isolated hepatocytes were prepared. Forward scatter values were determined for each preparation. (B) Hepatocytes (4×10⁵) from pregnant and nonpregnant mice were resuspended in 50 μl of PBS, loaded onto a hematocrit capillary, and centrifuged at 3000 g. (C) Mean hepatocyte volume was calculated for each mouse by measuring the total volume of cells and dividing by cell number.

FIGS. 7A-C show hepatocyte proliferation after delivery. Aged pregnant mice were hepatectomized at near term (n=5) or left untreated (n=4). After delivery, BrdU was administered through the drinking water for 6 weeks. The mice were then killed and their livers were examined histologically. Hepatocyte proliferation, indicated by BrdU incorporation, was determined by immunohistochemistry. Shown are mean proliferation values±s.e.m. (A) and representative photomicrographs of BrdU-stained liver sections (B and C).

FIG. 8 demonstrates that following hepatectomy, p53 (upper panel) and its target p21 (lower panel) are upregulated in nonpregnant mice. Liver sections from mice at the indicated days after hepatectomy were immunostained for p53 or p21. The extent of positive nuclei was assessed by two observers that were blinded to the treatment group. Values are mean±s.e.m. Nonpregnant mice, triangles; pregnant mice, circles.

FIGS. 9A-B show that the Akt/mTORC1 pathway mediates the hypertrophy module in regenerating livers of pregnant mice. (A) Western blot analyses of liver extracts from aged nonpregnant (N) and aged pregnant (P) mice at the indicated times after two-thirds partial hepatectomy; v=liver extracts from young mice treated with the PTEN inhibitor bpV(phen). P-Akt (Thr 308), p-4EB-1 (Thr 37/46) and p-4EB-1 (Ser 65), antibodies directed at phosphorylated Act and 4EBP-1, respectively; Akt, antibody against Akt; Tubulin, control (B). Representative images of livers from aged nonpregnant (upper panels) and aged pregnant (lower panels) mice harvested on day 3 and day 4 after surgery and subjected to triple immunofluorescence staining for phospho-4E-BP1 (red), BrdU (green) and E-cadherin (blue). Scale bars represent 20 μm.

FIG. 10 shows activation of the Akt/mTORC signaling pathway. Western blot analyses of liver extracts from aged nonpregnant and pregnant mice, 2 days after ⅔ partial hepatectomy.

FIGS. 11A-F show that the Akt/mTOR pathway controls the switch from the hyperplasia to the hypertrophy regeneration module. (A, top panel) Immunohistochemical staining for BrdU in vehicle- and rapamycin-treated aged pregnant mice 2 d after partial hepatectomy. Note the apparently paradoxical proliferation induced by the anti-proliferative drug rapamycin in the aged pregnant mice. Bars, 100 μm. P=0.04, Student's t-test. In the upper left panel, 2±1% were labeled; In the upper right panel, 25±8% were labeled; In the lower left panel, cell size was 527±2μ²; and in the lower right panel, cell size was 422±1μ² (A, Bottom panel) Immunofluorescence staining for E-cadherin (bright outline). Bars, 20 μm. Lower left panel, (B) Cell size distribution 2 d after surgery in hepatectomized livers of aged pregnant vehicle-treated (triangles), aged pregnant rapamycin-treated (circles), and aged nonpregnant untreated (diamonds) mice. Each data point is representative of at least three mice. (C) Immunohistochemical staining for BrdU of livers from young nonpregnant (NP), nonpregnant treated with bpV(phen) [NP+bpV(phen)] or pregnant (P) mice 4 d after partial hepatectomy and continuous BrdU administration. Bars, 100 μm. In the left panel, 92±1% were labeled; in the middle panel 8±2% was labeled; and in the lower right panel, 5±1% was labeled (D) Average proliferation indices 2 d after hepatectomy with continuous BrdU in drinking water in mice of the indicated treatment groups. (E) Kaplan-Meier plots depicting survival in aged pregnant (black continues line), aged nonpregnant (dotted line), old (gray continues line), and bpV(phen)-treated old (dashed line) mice. (F) Proposed model for the two modules of liver regeneration. In most situations, liver regeneration occurs via hyperplasia. Certain circumstances, such as pregnancy and pharmaceutical activation of the Akt/mTORC1 pathway [e.g., by treatment with bpV(phen) or leptin], favor the hypertrophy module. Aging affects the latter module much less strongly than it affects the hyperplasia module, where it results in impairment of liver function and significant mortality.

FIG. 12 depicts cell-size distribution, 4 days after surgery, in hepatectomized livers of young untreated (diamonds) and bpV(phen)-treated (triangles) nonpregnant mice. Each data point is representative of at least three mice.

FIGS. 13A-E suggest that leptin is a mediator of the pregnancy induced switch from the hyperplasia to the hypertrophy module. (A) Average percentage of BrdU-labeled hepatocytes in livers of pregnant and nonpregnant ob/ob mice 4 days after partial hepatectomy with continuous BrdU administration. (B-D) Livers from pregnant and nonpregnant ob/ob mice at day zero (diamonds) or day 4 (squares) after partial hepatectomy were stained with E cadherin (red); nuclei were stained with PI (green). Cell size was determined for at least 500 cells per mouse by an observer blinded to the treatment and genotype. Note that although some hypertrophy of hepatocytes does occur physiologically in ob/ob mice (compare pregnant and nonpregnant mice on day 0), there is no further hypertrophy after partial hepatectomy. (E) Western blot analyses of liver extracts from nonpregnant and pregnant ob/ob mice 4 days after 55% partial hepatectomy.

FIG. 14 shows that leptin activates the Akt pathway in H-35 cells (hepatocellular carcinoma cell line). Western blot analyses of hepatoma rat cells, that were under starvation for 16 h and then treated with: bpV(phen), leptin, IL-6 and combination of IL-6 and Leptin.

FIGS. 15A-D show that leptin is sufficient to induce reduction in proliferation and activation of the AKT pathway. (A) Experiment description—Mice were injected (S.C. 1 mg/kg body weight) with mouse leptin or saline two days before partial hepatectomy (P.H.), and until the end of the experiment, two days after the surgery. They received BrdU continuously in drinking water after the partial hepatectomy. (B) Percentage of BrdU-positive cells two days after two-thirds partial hepatectomy. BrdU incorporation into hepatocytes was assayed using immunohistochemistry. Ctrl, control; lep, leptin (C) Immunohistochemical staining for BrdU in the liver. (D) Phosphorylation of the 4E-BP protein as an indicator for AKT pathway activation. WT, wild type.

### DETAILED DESCRIPTION OF THE INVENTION

Organ and limb regeneration have fascinated humankind from the earliest days of science. In mammalians, accurate regeneration of an entire limb or organ does not occur. Instead, regenerative programs have evolved that result in reconstitution of organ function and mass, but do not accurately replace anatomy and cellular composition. Liver regeneration after partial hepatectomy is perhaps the best-studied mammalian model for such processes. In this model, the liver mass and function, but not its micro- and macroanatomy, are usually regenerated via proliferation of terminally differentiated hepatocytes.

In the clinical setting, liver regeneration is often desired after liver damage, either anatomical or functional, or both, for example after the removal of liver tumor or damage caused by hepatitis. However, regeneration is also desired after liver transplantation of a whole liver or a portion of a liver to a person who has had the liver removed. In the case of the whole liver, it can be considered damaged in the sense that it has been disconnected from its original environment.

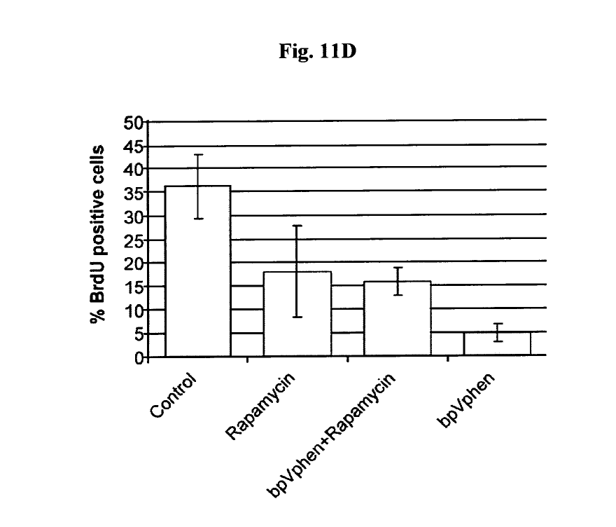

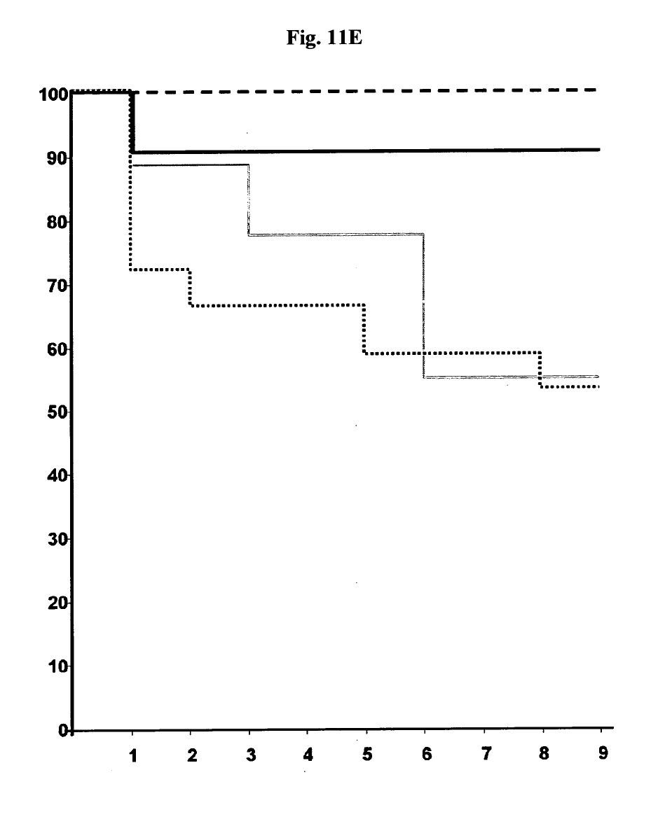

It has now been found in accordance with the present invention that there are two physiological modules for reconstitution of liver mass: hyperplasia (the primary module in nonpregnant mice) and hypertrophy (the primary module in pregnant mice). The latter module is activated in pregnant mice via signaling through the Akt/mTORC1 pathway (FIG. 11F).

mTOR (Mammalian Target of Rapamycin) is a 289-kDa serine/threonine protein kinase and a member of the PIKK (Phosphatidylinositol 3-Kinase-related Kinase) family. TOR proteins are evolutionarily conserved from yeast to human in the C-domain, with human, mouse, and rat mTOR proteins sharing 95% identity at the amino acid level. The human mTOR gene encodes a protein of 2549 amino acids with 42% and 45% sequence identity to yeast TOR1 and TOR2, respectively. mTOR functions as a central element in a signaling pathway involved in the control of cell growth and proliferation.

The mTOR pathway is regulated by a wide variety of cellular signals, including mitogenic growth factors, hormones such as insulin and leptin, nutrients (amino acids, glucose), cellular energy levels, and stress conditions. A principal pathway that signals through mTOR is the PI3K/Akt (v-Akt Murine Thymoma Viral Oncogene Homolog-1) signal transduction pathway, which is critically involved in the mediation of cell survival and proliferation. Signaling through the PI3K/Akt pathway is initiated by mitogenic stimuli from growth factors that bind receptors in the cell membrane. These receptors include IGFR (Insulin-like Growth Factor Receptor), PDGFR (Platelet-Derived Growth Factor Receptor), EGFR (Epidermal Growth Factor Receptor), and the Her family. The signal from the activated receptors is transferred directly to the PI3K/Akt pathway, or, alternatively, it can be activated through activated growth factor receptors that signal through oncogenic Ras. Phosphatidylinositol (3,4,5)-triphosphate (PIP3) and phosphatidic acid (PA) can activate mTOR via this signaling cascade.

It seems that the choice of regenerative module is critical for expression of the negative manifestations of aging. As is shown hereinafter, the hyperplasia module is negatively affected by aging, which delays restoration of liver function in old mice and results in a decrease in their ability to accommodate acute loss of liver mass. This may be due to accumulating damaged nuclei, resulting in a reduction in the pool of hepatocytes that can be recruited rapidly to the cycling pool. Our findings show that the hypertrophy regeneration module is less affected by aging; pharmacological activation of Akt in old organisms induces the hypertrophy module, thereby restoring the functional capacity for liver regeneration. Thus, our results suggest that a useful therapeutic approach to improve liver regeneration in the aged might involve activation of a regenerative module that is less sensitive to aging.

In certain embodiments, the activator used for activation of the regenerative module, i.e. for improving liver regeneration, increasing liver mass, or improving liver function, is leptin. The leptin may be human leptin or a non-human mammal leptin such as, but not limited to, ovine, rat, mouse, horse and pig leptin.

The term “leptin” as used herein refers not only to native leptin, but also to a fragment of leptin, an analog of leptin that is modified by substitution of one or more amino acid residues for a different amino acid residue, and a leptin, a leptin fragment or an analog modified with for example polyethylene glycol, all of which are themselves leptin agonists.

In one aspect, the present invention provides leptin for use in liver regeneration increasing liver mass, or improving liver function, or a combination thereof; i.e. leptin may be used for these purposes without being limited by mechanism. Human leptin is also known as FLJ94114, OB or OBS and can be identified by MIM: 164160 and ID: 3952. Mouse leptin is also known as ob or obese and can be identified by ID: 16846.

In certain embodiments, the activator or leptin is for use in regenerating damaged liver, increasing the mass of a damaged liver or improving function of the damaged liver, or a combination thereof. Furthermore, the AKT/mTOR pathway activator may be used in treating liver damaged due to surgical operation, for example removal of a tumor; injury; a disease; a pathological condition, or trauma.

The enhanced liver regeneration and/or increase in liver mass and/or improvement in function may be desired where a liver or liver section is implanted to a subject to replace the subject's damaged or malfunctioning liver.

Where the application is desired in order to improve regeneration or increase mass of a transplanted liver or liver section, the activator may be applied directly on the liver to be implanted while it is still ex-vivo, immediately during the operation to the liver recipient and/or several days post operation. Where liver regeneration is required due to planned removal of a part of the liver by surgery (for example due to tumor in the liver), or due to hepatitis, the period of administration can be divided to pre -operation and post-operation administration period. For example where the administration is for 4-5 days it is possible to administer the activator for 1-2 days prior to the operation and 3-4 additional days after the operation. Typically the subject being treated is an adult subject (above the age of 20).

The conditions that require liver regeneration include the following: a situation where a part of the liver is removed due to surgery; where liver is damaged due to trauma; or where liver is damaged due to a disease process (without being removed, e.g. hepatitis) that caused significant degree of acute liver dysfunction. Thus, the disease or condition that may be the cause for damage of the liver is selected from: acute liver damage caused by exposure to alcohol, e.g. steatosis, alcoholic hepatitis or cirrhosis; acute viral hepatitis, such as hepatitis type A; a metabolic disease resulting in abnormal storage of copper, such as Wilson's disease, or iron (hemochromatosis); acute liver damage caused by exposure to drugs or toxins, acute hepatitis caused by autoimmune processes, such as autoimmune hepatitis; or acute liver damage caused by obesity or other causes of acute steatohepatitis.

In certain embodiments, the activator (or leptin) is for local or systemic administration, including, but not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes.

Local administration may be achieved by direct application of the activator to the operated liver (immediately after removal of the damaged region), or alternatively by administration to the liver (pre- or post-operation) by the portal vein. In certain embodiments, the activator is applied by administering locally to the liver a therapeutically effective amount of leptin.

The activator or leptin may by administered for a period of up to 7 days, up to 14 days or up to 30 days, i.e. for a period of 1, 2, 3, 4, 5, 6, 7, 8, 9, 10, 12, 14, 21 or 30 days, in particular 7 days.

In certain embodiments, a therapeutically efficient amount of activator or leptin is administered to the person in need of enhanced liver regeneration and/or increase in liver mass and/or improvement in liver function.

As shown below in the Examples, a dose of 1 mg leptin/kg body weight was sufficient to induce reduction in proliferation of hepatocytes and activation of the AKT pathway after partial hepatectomy in mice. An expected approximate equivalent dose for administration to a human can be calculated using known formulas (e.g. Reagan-Show et al. (2007) Dose translation from animal to human studies revisited. The FASEB Journal 22:659-661) to be 0.61 mg/kg or about 36 mg for a 60 kg adult and about 60 mg for a 100 kg adult. Thus, the therapeutically effective dose of leptin for administration in a human should be in the range of about 0.4 mg to about 60 mg/day.

In certain embodiments, the activator according to the present invention consists of a combination of two or more activators of the AKT/mTOR pathway.

Pharmaceutical compositions for use in accordance with the present invention may be formulated in conventional manner using one or more physiologically acceptable carriers or excipients. The carrier(s) must be “acceptable” in the sense of being compatible with the other ingredients of the composition and not deleterious to the recipient thereof.

The term “increased regeneration” or “improved regeneration” as used herein is manifested by a shorter period needed to reach the final liver mass or increased final liver mass (for example as determined by MRI), or both, to an increase in the final mass and the rate of reaching that mass, as compared to an untreated control.

The regeneration should be functional—resulting in an improvement of liver functions evident by higher levels in the serum of proteins that are produced by the liver, such as various coagulation factors, as compared to untreated control. In particular, liver function or integrity may be assessed by measuring any of a number of parameters as is well known in the art; for example, prolonged serum prothrombin time (blood coagulation) is a sign of damaged liver; albumin levels are decreased in chronic liver disease; alkaline phosphatase levels in plasma rise with large bile duct obstruction, intrahepatic cholestasis or infiltrative diseases of the liver; increased total bilirubin may be a sign of problems in the liver; gamma glutamyl transpeptidase (GGT) may be elevated with even minor, sub-clinical levels of liver dysfunction; 5′ nucleotidase levels reflect cholestasis or damage to the intra or extrahepatic biliary system; or liver glucose production is reduced in a damaged liver.

The term “activator of the AKT/mTOR pathway” as used herein, refers to any agent that may be a small chemical molecule, such as an amino acid or nucleic acid based compound or an agonist of one or more of the many receptors signaling via this pathway (see above), that results in the activity of mTOR or its downstream targets that induce hepatocyte hypertrophy. The agent may work directly on AKT by increasing its amount (on the protein or mRNA level, or both) or by increasing AKT activity for example by regulating the phosphorylation pattern of AKT to increase its activity. Alternatively, the agent may work directly on mTOR by increasing its amount (on the mRNA and/or protein level) or by increasing mTOR activity, for example by regulating the phosphorylation pattern of mTOR to increase its activity.

The activator may also work upstream from the AKT for example on the PIP2 hydrolyzing enzyme phospholipase C, phosphatase and tensin homolog (PTEN), Phosphatidylinositol 3-kinase (PI3K), Phospholipid-Dependent Kinase-1 (PDK-1) or PDK-2 in a direction that increases AKT activity. As shown hereinafter, a specific example of an activator is an inhibitor of the PTEN phosphatase and more specifically of the inhibitors bpV(phen) or V(oh)pic.

The drug leptin may be included alone or in combination in the treatment as we have shown that in vivo leptin is necessary for hypertrophy based increase in liver mass after hepatectomy in pregnant mice.

The invention will now be illustrated by the following non-limiting examples:

### EXAMPLES

### Materials and Methods.

Animal studies and tissue preparation. All animal experiments were performed in accordance with the guidelines of the Institutional Committee for the Use of Animals for Research (IACUC). Mice aged 18 months or older (‘old’ mice) were either purchased from the National Institute of Aging and from Charles River Laboratories or maintained up to the required age in the Specific Pathogen Free (SPF) animal facility at our institution. Mice aged 10-12 months (‘aged’ mice) and 8-week-old ob/ob mice were purchased from Harlan Laboratories. The genetic background of all mice was c57 Black. Pregnancy in ob/ob mice was induced as described previously (Malik et al., 2001). Pseudopregnancy was induced by mating females with vasectomized males and observing for the presence of vaginal plugs. Progesterone levels were measured to verify pseudopregnancy induction (Shiotani et al., 1993). For rapamycin treatment, mice were injected intraperitoneally (i.p.) for 3 consecutive days with rapamycin (0.2 mg/kg body weight diluted in DMSO; LC Laboratories), starting 3 h before the hepatectomy. For PTEN inhibition, bpV(phen) (3.3 μg/g body weight diluted in normal saline; Alexis Biochemicals) was administered i.p. on the day before hepatectomy and once a day for 3 days thereafter. BrdU (100 μl/10 g body weight; Amersham) was injected i.p. at the indicated times before the mice were killed. When indicated, BrdU (#B5002; Sigma) was added to the drinking water (0.8 mg/ml). The mice were allowed to drink ad libitum. For all experiments mice were killed by cervical dislocation. In some cases, a liver sample was removed and ‘snap-frozen’ for protein and RNA analyses. Livers were removed, weighed, photographed, and fixed in formalin overnight, and the next day the entire liver was embedded in paraffin. For measurement of serum prothrombin time, sodium citrate (0.105 M) was added immediately to the blood sample at a ratio of 1:10 (v/v), and values were recorded with a Beckman Coulter ACL 9000 Coagulation Analyzer according to the manufacturer's instructions. For ⅔ partial hepatectomy, virgin or pregnant mice (16-18 days post-coitum unless otherwise specified) were anesthetized, and the median and left lateral lobes of the liver were removed, as described (Ben Moshe et al., 2007).

Measurement of cell size. Sections (5 μm thick) were prepared from formalin-fixed, paraffin-embedded livers. Slides were stained with anti E-cadherin antibodies. Digital images were obtained using a Nikon 90i confocal microscope at 400× magnification. The numbers of pixels in 100-300 individual hepatocytes for each mouse were scored with ImageJ (NIH) by an observer who was blinded to the treatment group. For FACS analysis, hepatocytes were isolated as described previously (Pikarsky et al., 2004) and forward scatter values were used as an indicator of cell size. To measure ‘hepatocrit’, equal numbers of isolated hepatocytes suspended in 50 μl of PBS were loaded onto a hematocrit capillary and centrifuged at 3000 g for 10 min. The height of the hepatocyte column was divided by the total height. Mean hepatocyte volume was calculated by dividing the hepatocyte volume by the number of hepatocytes.

Antibodies. Primary antibodies against the following proteins and chemicals were used: BrdU (cat #MS-1058) from Thermo Scientific; Akt (cat #9272), phosphoAkt Thr308 (cat #9275), phospho-4E-BP1 Ser65 (cat #9451), and phospho-4E-BP1 Thr37/46 (cat #2855), all from Cell Signaling; E-cadherin (cat #610182) from Becton-Dickinson; and tubulin (cat #T9026) from Sigma.

Proliferation index. The percentage of BrdU-positive hepatocyte nuclei was assessed using the Kisight module of the Ariol SL 50® automated scanning microscope and image analysis system, according to the manufacturer's instructions. The same gating parameters were used for all sections. Ten fields in each liver were scored and the average percentage was calculated.

Locomotor activity. This was monitored using a photocell cage, 43.2 cm×43.2 cm (Med Associates), with 16 beam I/R arrays located along each wall of the box. Data are mean values of the total numbers of beam breaks (representing horizontal activity).

MRI analysis. MRI was performed on a horizontal 4.7T Biospec spectrometer (Bruker Medical), using a 3.5 cm birdcage coil. Mice were anesthetized (30 mg/kg pentobarbital, i.p.) and placed supine with the liver located at the center of the coil. Liver volumes were determined from multi-slice coronal and axial T₁-weighted fast spin-echo images covering the entire liver (repetition time, 400 ms; echo time, 18 ms; slice thickness, 1 mm; field of view, 5 cm (coronal) and 3.4 cm (axial) using a 256×256 matrix). An observer who was blinded to the treatment group outlined the liver boundaries visualized in each slice, using image processing software (NIH image). To convert the pixel count to an area it was multiplied by the factor [(field of view)²×(matrix)²]. Total liver volume was calculated as the summed area of all slices, multiplied by the slice thickness. The post-hepatectomy liver volume of each mouse was expressed as a percentage of the preoperative volume (Ben Moshe et al., 2007).

Western blot analysis. Liver samples were homogenized in cell culture lysis reagent (Promega) with a Polytron homogenizer. Tissue lysates containing 100 μg protein were separated by 12% SDS-PAGE, and assessed by Western blot analysis by means of sequential probing with the relevant primary antibody and a relevant anti-IgG conjugated to horseradish peroxidase (Jackson ImmunoResearch).

Immunohistochemistry and immunofluorescence. For BrdU immunostaining, sections (5 μM) were dewaxed and hydrated through graded ethanols, cooked in 25 mM citrate buffer pH 6.0 in a pressure cooker at 115° C. for 3 min (decloaking chamber, Biocare Medical), and then transferred to boiling deionized water and allowed to cool for 20 min. After treatment for 5 min in 3% H₂O₂, slides were incubated with mouse monoclonal anti-BrdU antibodies diluted 1:200 in CAS-Block (Zymed) overnight at 4° C., washed three times with Optimax (BioGenex-HK583), incubated for 30 min with anti-mouse Envision⁺ K4007 (Dakocytomation), and developed with 3,3′-diaminobenzidine (Dakocytomation) for 15 min.

For measurement of E-cadherin immunofluorescence, sections (5 μM) were dewaxed and hydrated through graded ethanols, cooked in 10 mM Tris/0.5 mM EGTA at pH 9.0 in a pressure cooker at 115° C. for 3 min (decloaking chamber), and then transferred to boiling deionized water and allowed to cool for 20 min. Slides were then incubated with mouse monoclonal anti-E cadherin antibodies diluted 1:50 in CAS-Block (Zymed) overnight at 4° C., and revealed with Cy5-labeled secondary antibodies. For triple staining, the same antigen retrieval procedure was employed and the relevant primary and secondary antibodies were added.

Statistics. All values are means±s.e.m. The indicated statistical tests were performed using StatXact software. Where applicable, all tests were two-sided.

### EXAMPLE 1

### In Aged Mice, the Rate of Liver Volume Gain, Liver Function and Survival after Partial Hepatectomy were all Markedly Improved by Pregnancy

It was shown recently that heterochronic parabiosis (connecting the circulations of a young and an old mouse) can restore the regenerative capacity of striated muscle in old mice and increase the basic rate of cell proliferation in aged livers (Conboy et al. 2005). The effect of heterochronic parabiosis on liver regeneration was not studied. Pregnancy can be viewed as a natural state akin to parabiosis, where organisms partly share blood systems—in this case, an adult organism (the pregnant mother) is exposed to extremely young organisms (the fetuses).

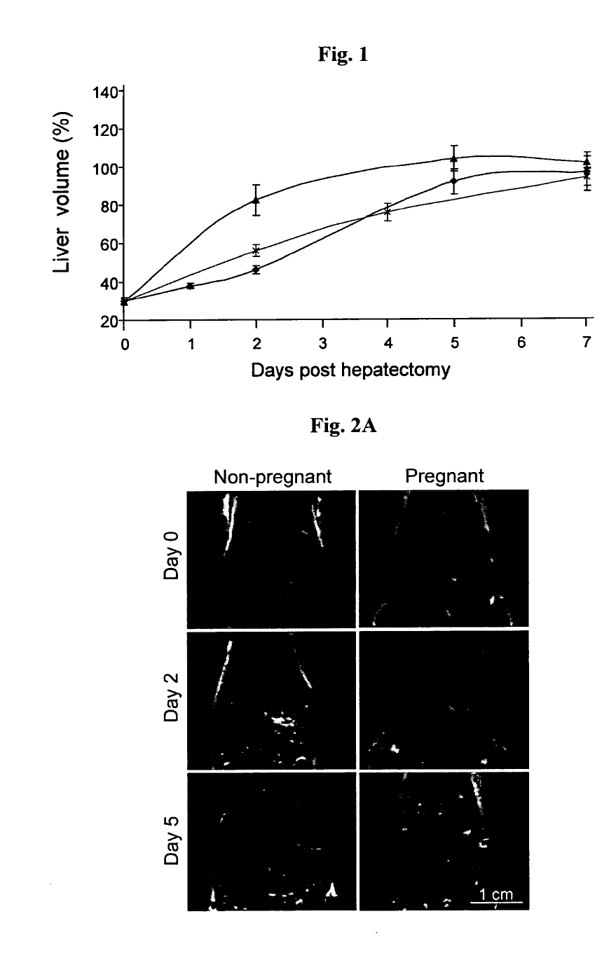

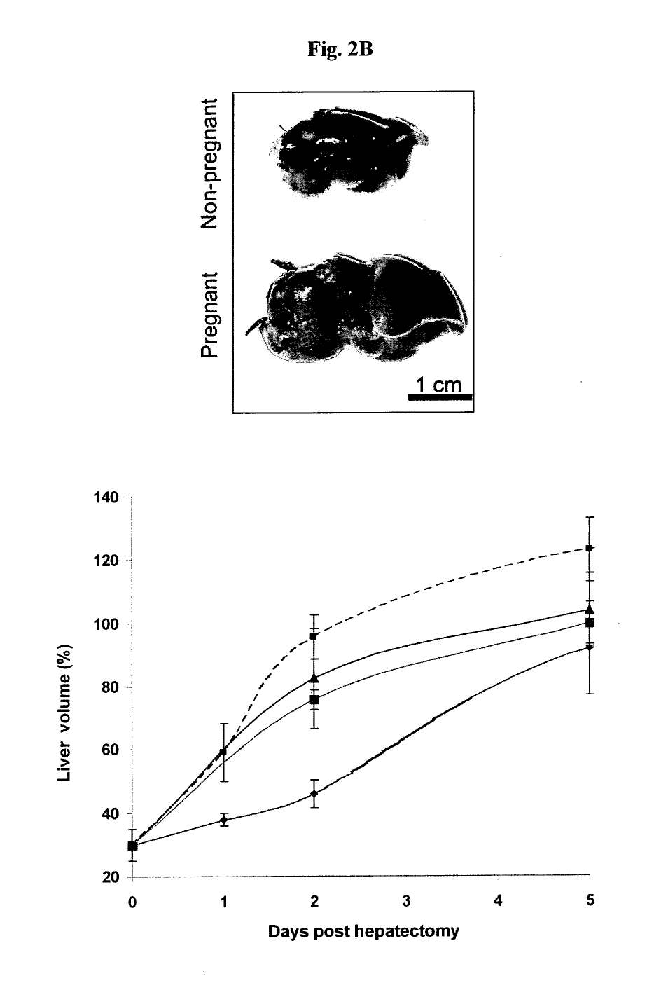

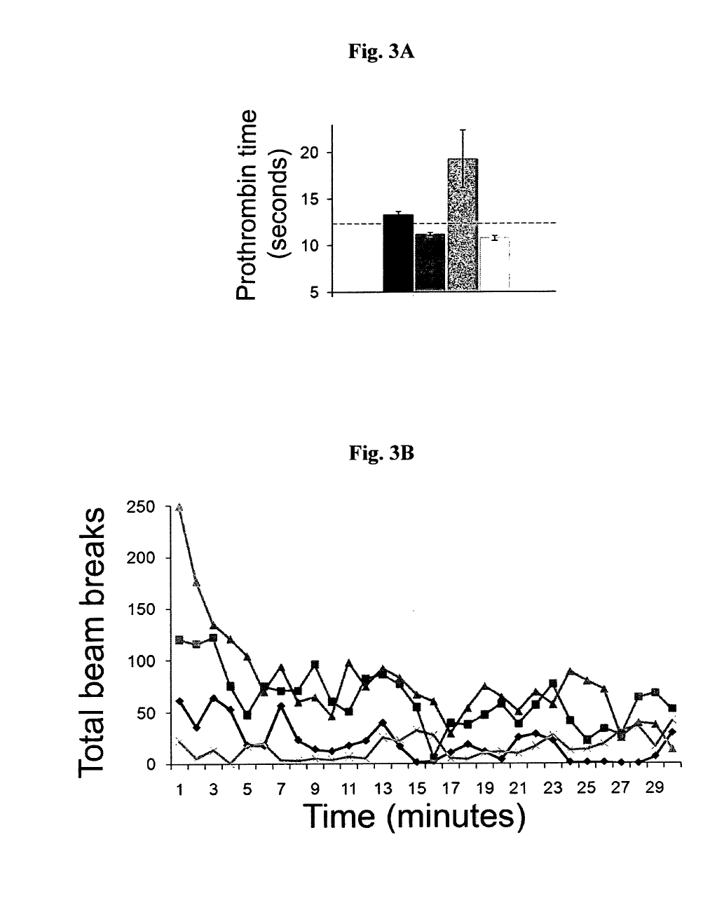

Pregnancy in mice increases baseline proliferation of pancreatic β cells and neurons and enhances post-injury remyelination (Karnik et al. 2007). We therefore set out to examine whether pregnancy also attenuates the age-related decline in regenerative capacity of the liver. Using serial magnetic resonance imaging (MRI), which accurately measures liver volume (Inderbitzin et al. 2004; Ben Moshe et al. 2007), we analyzed the process of liver regeneration after two-thirds partial hepatectomy in nonpregnant and near-term pregnant 3-mo-old (hereafter “young”) and 10- to 12-mo-old (“aged”) mice. This procedure, which has been shown before to accurately measure liver volume (Inderbitzin et al. 2004; Ben Moshe et al. 2007), allowed us to follow single mice along the regeneration process. In the nonpregnant groups, 2 d after surgery, the total liver volume (mean 6 SEM) regenerated to 82%±8% of the original size in young mice, while in aged mice, the liver regenerated to only 46%. In contrast, liver regeneration in aged pregnant mice was dramatically more efficient, with 96%±3% of the liver volume restored within 2 d (FIGS. 2A-C). In the aged mice, blood coagulation (indicative of the liver's synthetic capacity) was pathological in the nonpregnant group, but within normal limits in the pregnant group (FIG. 3A). Similarly, whereas aged nonpregnant mice were lethargic after surgery, their pregnant counterparts were relatively active (FIG. 3B). Post-hepatectomy mortality in aged mice declined from 47% (nine out of 19) in the nonpregnant group to 9% (two out of 22; P=0.003, Fisher's exact test) in the pregnant group (FIG. 11E). Thus, in the aged mice, the rate of liver volume gain, liver function, and, most importantly, survival after partial hepatectomy were all markedly improved by pregnancy.

### EXAMPLE 2

### The Restored Capacity of the Aged Liver for Regeneration in Aged Pregnant Mice is a Function of Cell Growth Rather than Cell Proliferation

Liver regeneration normally begins with a priming phase, which is followed by a spurt of regeneration during which most of the hepatocytes enter the cell cycle (Michalopoulos 2007). We postulated that pregnancy in aged mice enhances liver regeneration by shortening the priming phase or by recruiting a larger number of hepatocytes into the cell cycle. To test this hypothesis, we injected nonpregnant and pregnant mice with the thymidine analog 5-bromo-2-deoxyuridine(BrdU) at several time points after partial hepatectomy and assayed its incorporation into hepatocytes using immunohistochemistry.

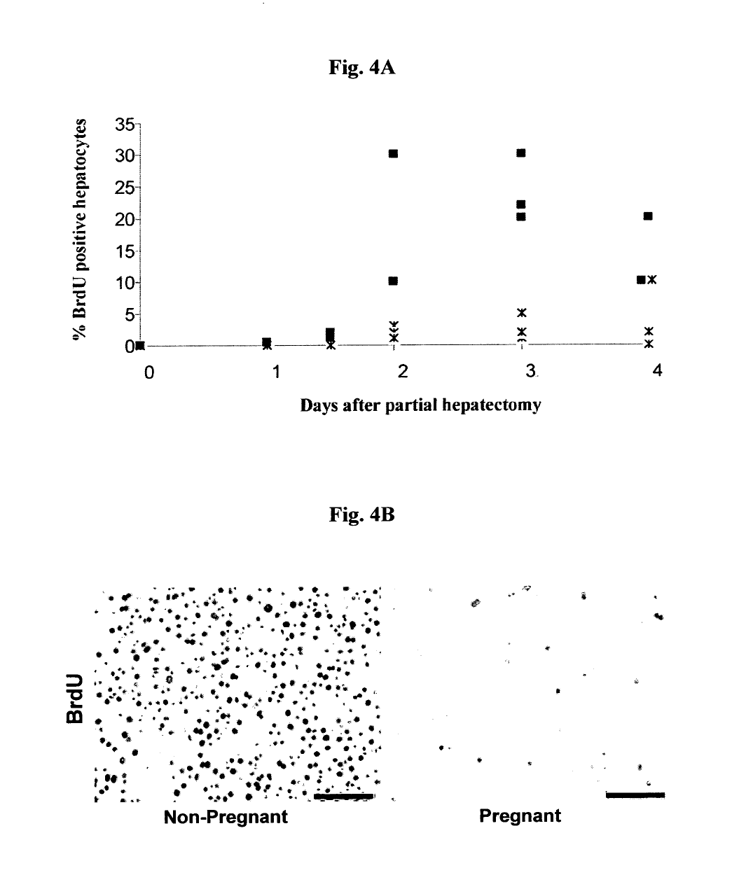

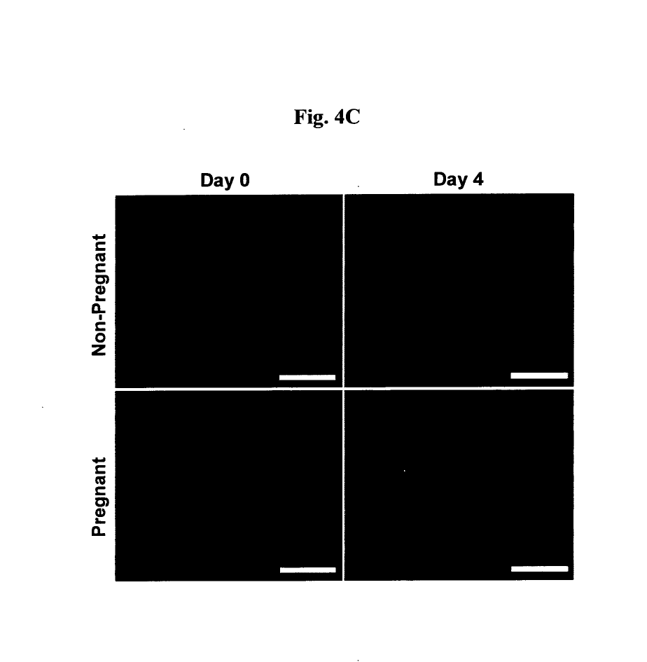

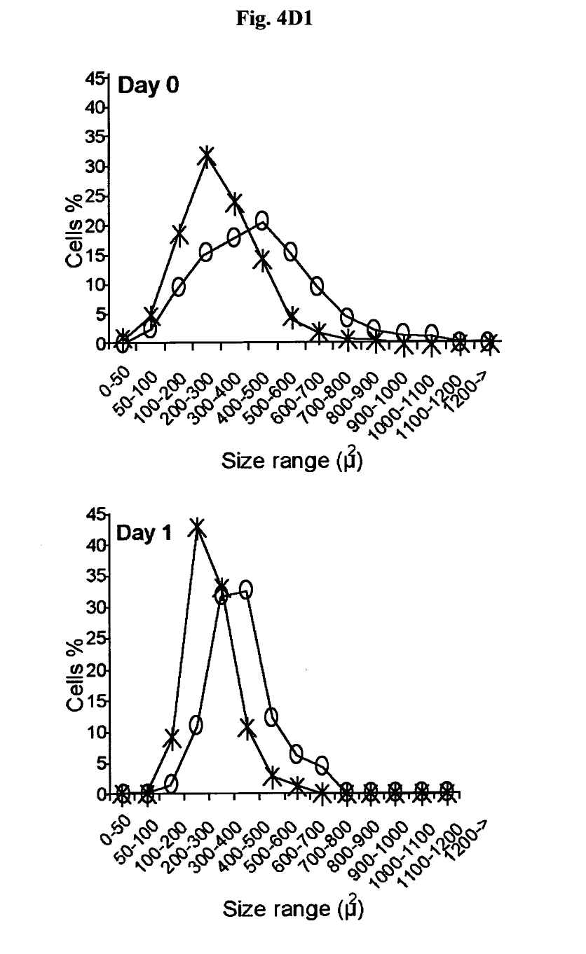

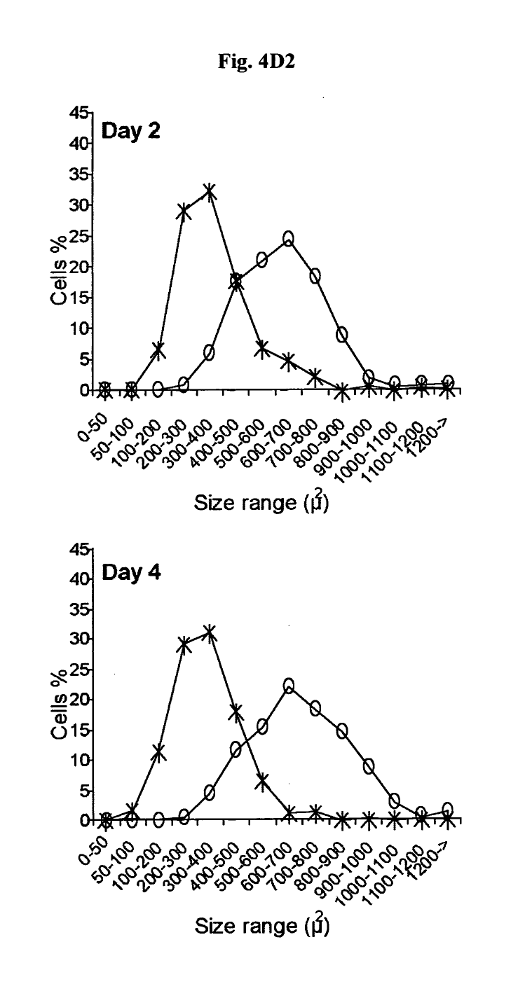

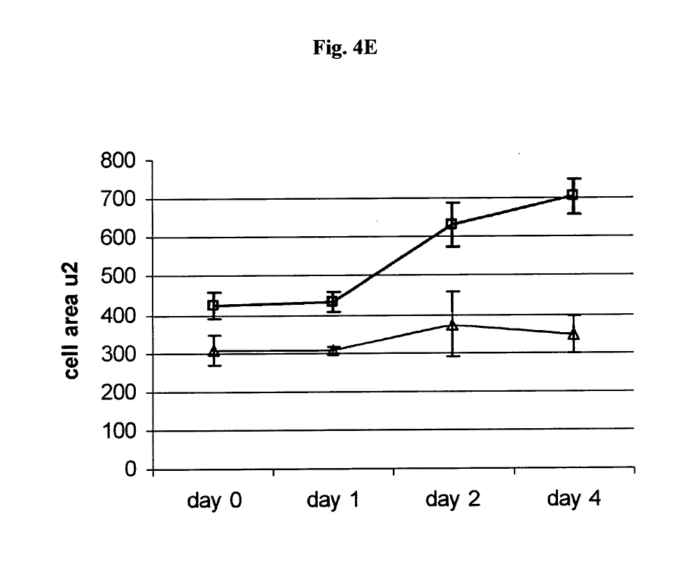

As expected, brisk proliferation occurred in the nonpregnant group between 48 and 96 h post-hepatectomy (FIG. 4A). Surprisingly, in the pregnant group, hardly any BrdU-labeled hepatocytes were observed at any of the time points measured. To rule out the possibility that we missed a specific time point at which hepatocytes in pregnant mice enter the S phase, we administered BrdU in the drinking water from the time of partial hepatectomy until 4 d after the surgical procedure, when the mice were killed. This would ensure that any hepatocytes entering the S phase during that 4-d period would be labeled with BrdU. This analysis also showed that very few hepatocytes in the pregnant mice had incorporated BrdU (6%±4% in the pregnant mice compared with 83%±7% in the nonpregnant mice; P=0.002, Student's t-test) (FIG. 4B). Pregnancy also affected liver regeneration in young mice, in which BrdU incorporation rates of 92%±1% and 5%±1% were recorded in the nonpregnant and pregnant groups, respectively (P<0.0001, Students's t-test).

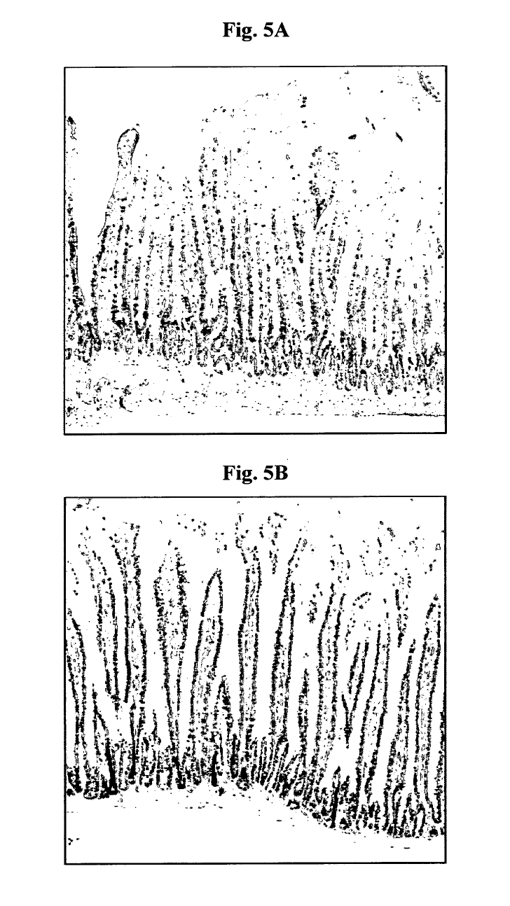

To rule out the possibility that pregnancy influenced BrdU labeling, small bowel samples were immunostained together with liver specimens on the same slide. Unlike liver sections, small bowel cells were clearly BrdU-labeled to the same extent, indicating that differential BrdU incorporation or metabolism cannot explain the observed differences (FIGS. 5A-B).

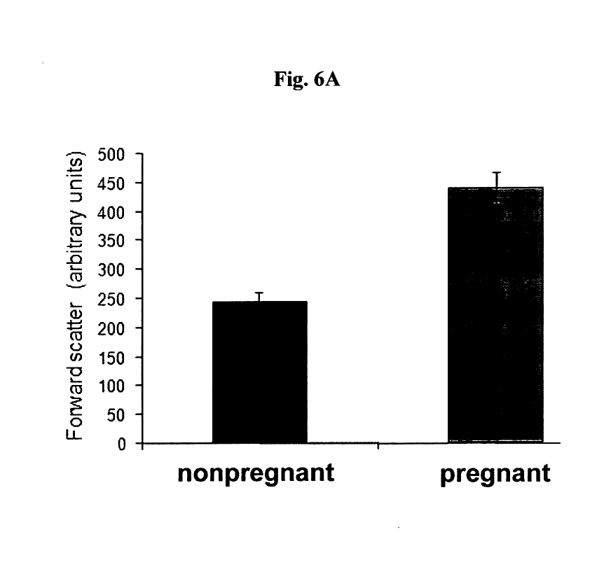

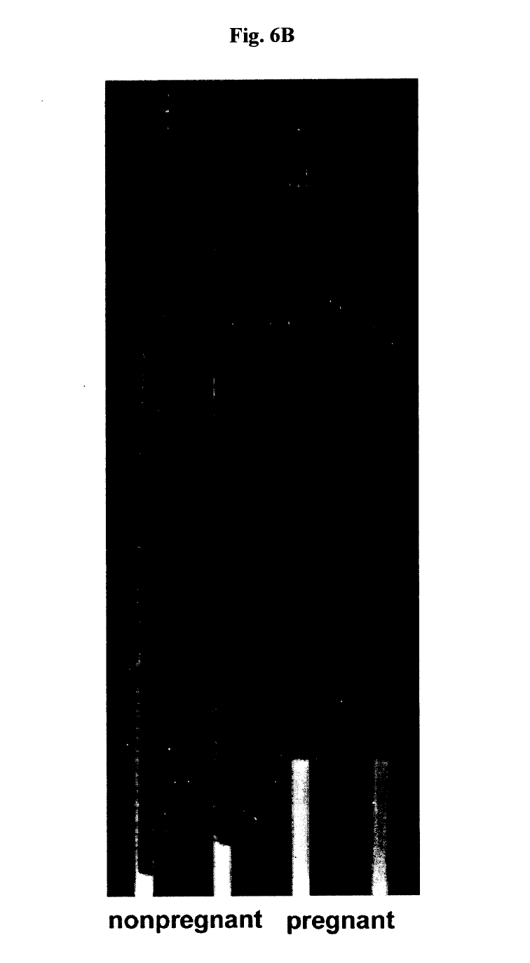

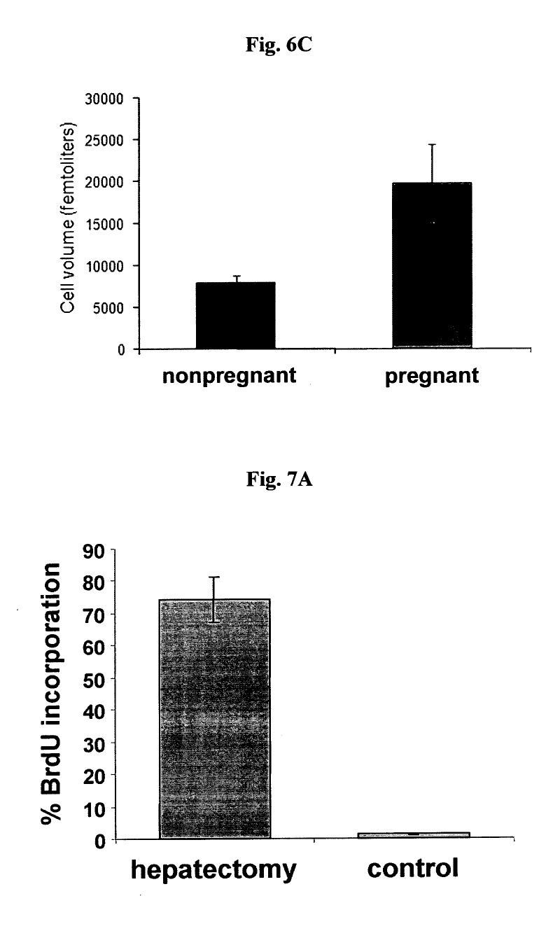

We therefore postulated that the restored capacity of the aged liver for regeneration in aged pregnant mice is a function of cell growth rather than cell proliferation. Indeed, while in nonpregnant aged mice a 13% increase in the average hepatocyte cross-sectional area was observed after partial hepatectomy, in pregnant aged mice this increase was 66% (FIGS. 4C-E). Both FACS and “hepatocrit” analyses of hepatectomized aged mice confirmed that hepatocytes isolated from the pregnant group were larger than those from the nonpregnant group (FIGS. 6A-B). Comparing the extent of proliferation and hypertrophy of nonpregnant, mid-pregnant, and late pregnant mice showed that the hypertrophy module gradually takes dominance during pregnancy (Table 1).

TABLE 1
Proliferation in non-pregnant, mid-pregnant, and late pregnant mice

| | non- | pseudo- | mid- | Late- |
|---|---|---|---|---|
| | pregnant | pregnant | pregnancy | pregnancy |
| BrdU incorporation (%) | 92 ± 1 | 59 ± 10 | 60 ± 6 | 5 ± 1 |
| Cell size (μ²) | 258 ± 2 | 348 ± 3 | 346 ± 3 | 663 ± 31 |

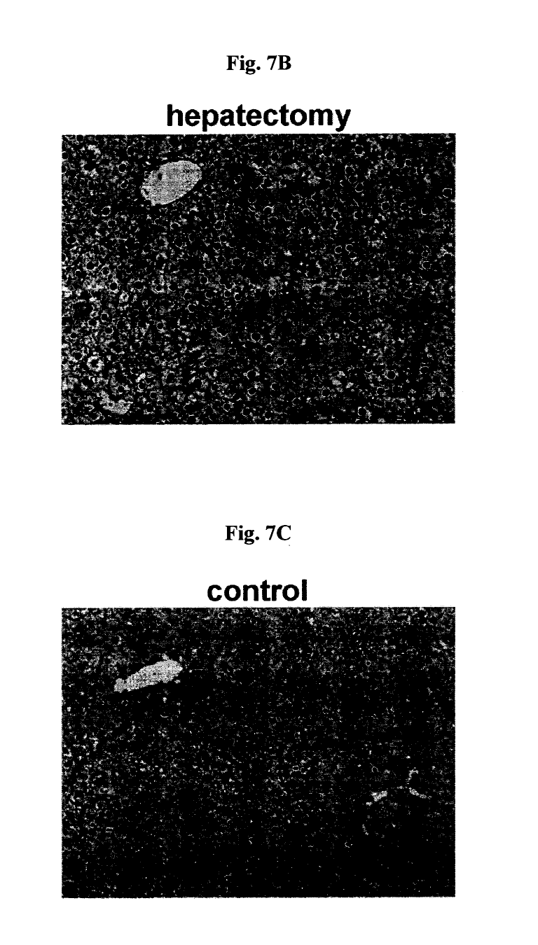

To study the fate of the hypertrophied hepatocytes, we subjected aged, late pregnant mice to partial hepatectomy, and this time administered BrdU in the drinking water only after delivery, 5 days after partial hepatectomy. Control mice were nonhepatectomized aged pregnant mice. Interestingly, after delivery, the hypertrophic hepatocytes that are generated in pregnant hepatectomized mice undergo considerable proliferative activity (FIGS. 7A-C). This suggests that pregnancy-related hypertrophy is maintained by a substance that is modulated continuously during pregnancy (either up-regulated or downregulated), yet returns to the nonpregnant levels after delivery.

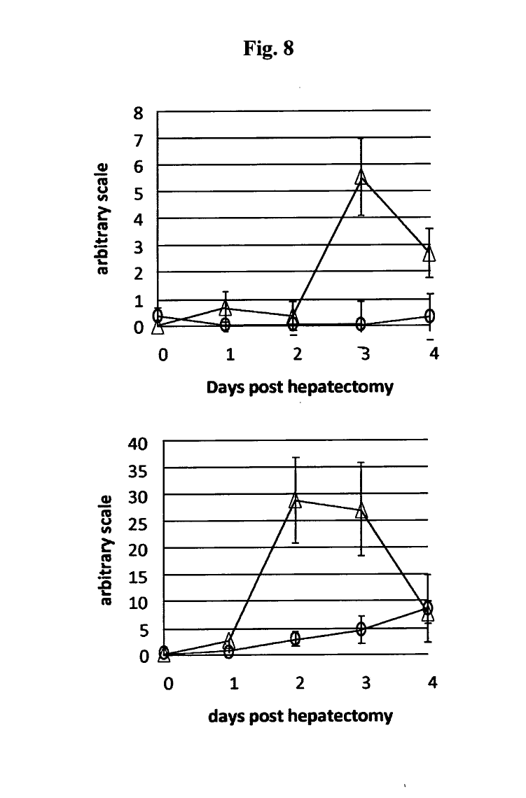

We immunostained liver sections for the cell cycle regulators p53, p21, and p27. This analysis indicated that, whereas levels of p27 did not differ between nonpregnant and pregnant mice (data not shown), both p53 and p21 are up-regulated after hepatectomy in nonpregnant mice but not in pregnant mice. This suggests that the up-regulation of these cell cycle inhibitors occurs in response to hepatocyte proliferation, and thus is absent from the pregnant mice (FIG. 8). Taken together, these findings indicate that, during pregnancy, hypertrophy, rather than proliferation, is the main mechanism by which the liver regains its volume and function.

### EXAMPLE 3

### What is the Source of the Hypertrophy-Inducing Factor?

To distinguish the putative roles of circulating maternal hormones from other physiological signals emanating directly from the embryo or in response to implantation, we mated young females with vasectomized males, which results in pseudopregnancy—a transient alteration of maternal pituitary and ovarian steroid hormones that mimics the changes during the first half of normal gestation (Erskine 1998). A similar decrease in post-hepatectomy proliferation and increase in cell size were noted in the pseudopregnant and midpregnant mice compared with the nonpregnant mice, albeit smaller than the effect of late pregnancy (Table 1). These results suggest that at least part of the effect of pregnancy on liver regeneration can be attributed to maternally derived factors. Taken together, the above findings confirmed that, in aged pregnant mice, post-hepatectomy liver regeneration results largely from hepatocyte hypertrophy.

Slight liver growth as a function of hypertrophy was shown to occur in pregnancy (Hollister et al., 1987). Restoration of liver mass after partial hepatectomy was shown to occur in several situations, such as after treatment with dexamethasone or 5-fluorouracil (Nagy et al., 2001), in deficiency of STAT3 (Haga et al., 2005) or Skp2 (Minamishima et al., 2002), and after γ-irradiation (Michalopoulos and DeFrances, 1997), indicating that hyperplasia and hypertrophy are two alternative modules for liver regeneration.

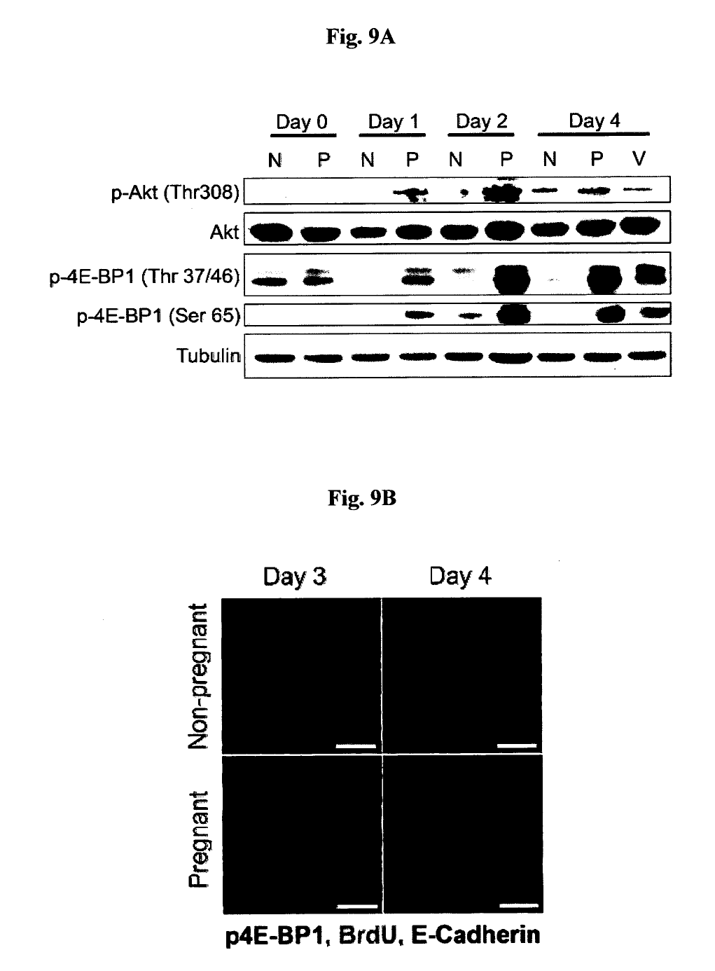

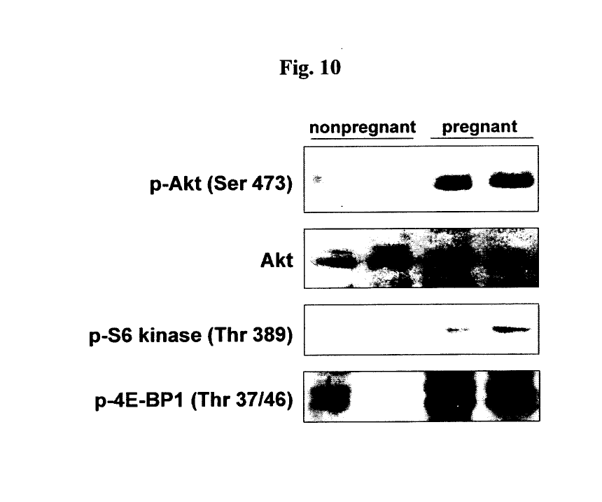

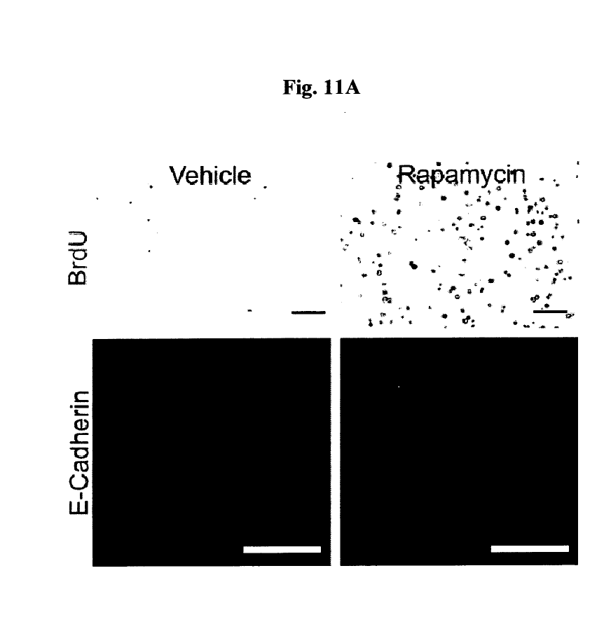

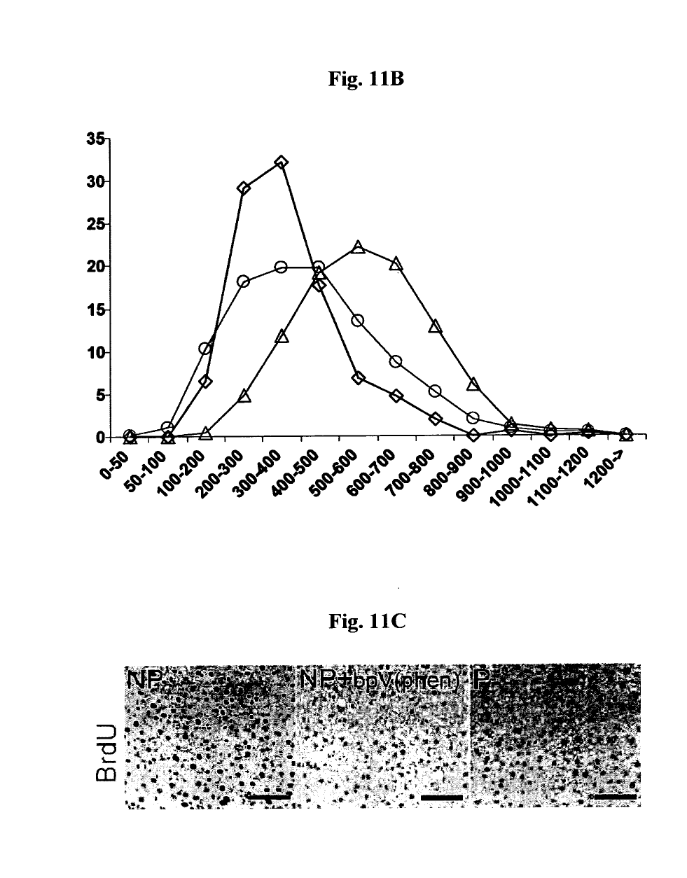

Our results provide novel evidence that a physiological condition—i.e., pregnancy—causes a switch from proliferation-based liver regeneration to a regeneration process mediated by cell growth. The Akt/mTORC1 pathway is a key mediator of cell growth in many cellular systems, including that of the liver (Mullany et al., 2007; Haga et al., 2009). We therefore examined whether pregnancy influences components of this pathway. Western immunoblotting of liver extracts revealed that, on post-hepatectomy days 1, 2, and 4, phosphorylation of Akt, S6 kinase, and 4E-BP1 were markedly increased in pregnant but not in nonpregnant mice (FIG. 9A; FIG. 10). Earlier (6 h after hepatectomy), Akt phosphorylation was increased in both groups, but was higher in the pregnant mice (data not shown). The above findings were confirmed by costaining for BrdU and phospho-4E-BP1 (FIG. 9B).

To assess the functional significance of Akt/mTORC1 signaling for liver regeneration in pregnant mice, we treated mice with the mTORC1 inhibitor rapamycin. This treatment resulted in a significant increase in the hepatocyte proliferation rate in pregnant mice (25%±8% compared with 2%±1%; P=0.04, Student's t-test) (FIG. 11A), concomitantly with elimination of pregnancy-induced cell growth (FIG. 11B). These findings differ markedly from numerous in vitro and in vivo systems demonstrating a strong anti-proliferative response evoked by rapamycin in other situations (Sanders et al., 2008). These results suggest that the Akt/mTORC1 pathway is a key determinant of hepatocyte hypertrophy in regenerating livers of pregnant mice.

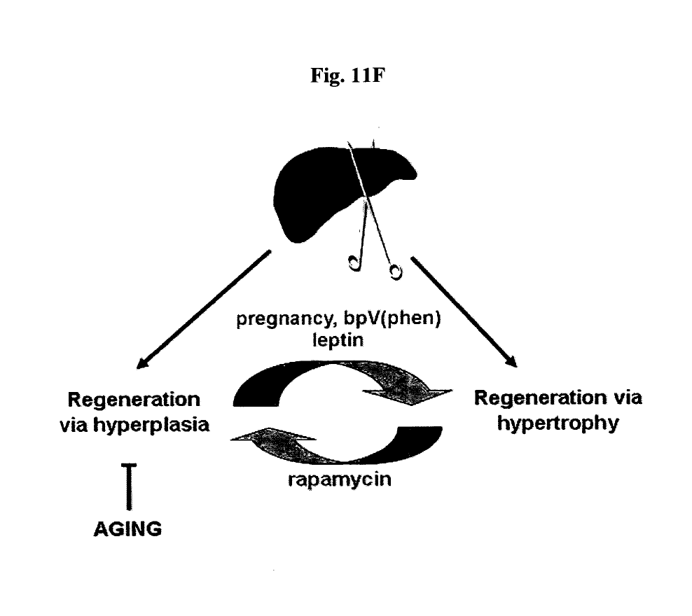

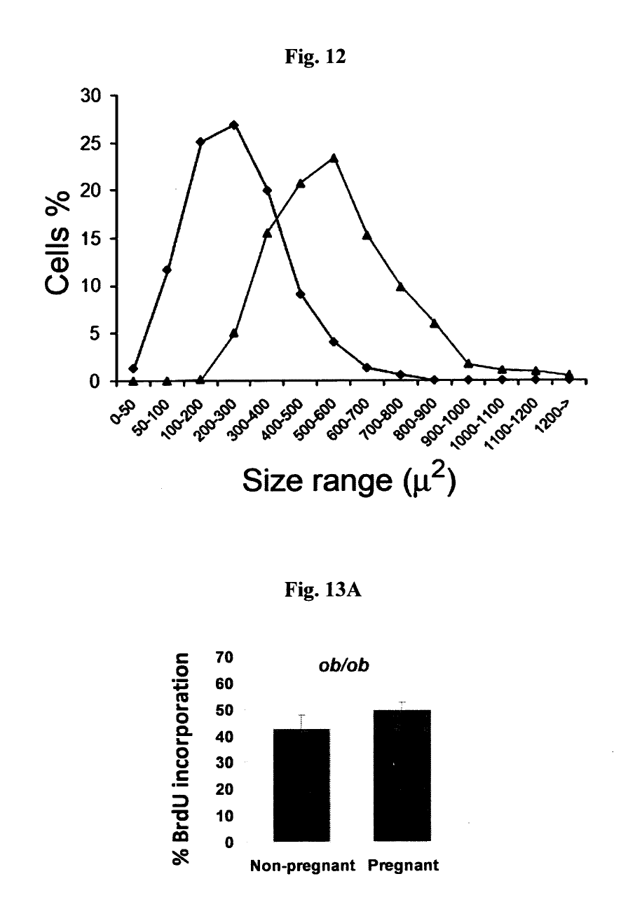

Can this switch in the liver regeneration module from hyperplasia to hypertrophy explain the observed improvement in the regenerative capacity of aged pregnant mice? If so, tilting the balance toward hypertrophy should improve liver regenerative capacity in old nonpregnant mice as well. We postulated that activation of the Akt/mTORC1 pathway in such mice might suffice to favor the hypertrophy pathway. To test this hypothesis, we first treated young nonpregnant mice with the phosphatase and tensin homolog (PTEN) inhibitor bisperoxovanadium 1,10-phenanthroline (bpV(phen)) before subjecting them to partial hepatectomy. Western blot analysis of phosphorylated Akt and 4E-BP1 confirmed that bpV(phen) treatment activates the Akt/mTORC1 pathway (FIG. 9A). Immunohistochemical analysis disclosed that liver regeneration in the bpV(phen)-treated mice proceeds via hypertrophy, as indicated by the low proliferation index and growth of 115% in the mean cross-sectional area (FIG. 11C; FIG. 12), indicating that bpV(phen) treatment of nonpregnant young mice suffices to activate the hypertrophy regeneration module. To support the possibility that the effect of bpV(phen) is mediated via mTORC1 signaling, we compared post-hepatectomy proliferation rates in control mice, mice treated with bpV(phen) alone, rapamycine alone, or combined treatment with bpV(phen) and rapamycine.

Whereas rapamycin treatment alone reduced post-hepatectomy proliferation rate (Sanders et al., 2008), the bpV(phen)-induced switch from hyperplasia to hypertropy was clearly blocked by rapamycin treatment (FIG. 11D). Haga et al., (2009) have shown recently that genetic activation of Akt via PDK1 contributes to liver regeneration by regulating cell size, further supporting the possibility that the bpV(phen) effect is mediated via Akt activation. This enabled us to test our hypothesis that this module may restore the regenerative capacity in old mice. To determine whether activation of the hypertrophy module by bpV(phen) is sufficient to restore the liver's regenerative capacity in old mice, we subjected female mice aged 18-24 mo to partial hepatectomy without (control) or with bpV(phen) treatment. Post-hepatectomy blood coagulation and locomotor activity tests confirmed that bpV(phen) treatment resulted in a significant improvement in recovery from partial hepatectomy compared with nontreated old mice (FIG. 11E; FIGS. 3A-B). Remarkably, the mortality rate in the bpV(phen)-treated old mice was zero out of nine, compared with four out of nine in the control group (P=0.014, Fisher's exact test) (FIG. 11E).

We wished to test whether the proliferation to hypertrophy switch during pregnancy was dependent on circulating maternal hormones or, rather, on embryo-derived factors. To do this, we compared both hepatocyte proliferation rates and hepatocyte sizes following partial hepatectomy in young nonpregnant mice, pseudopregnant mice (generated by mating young females with vasectomized males), and pregnant mice at mid- and late pregnancy. Compared with nonpregnant mice, pseudopregnant and mid-pregnant mice showed decreased post-hepatectomy proliferation and increased cell size, albeit to a lesser extent than late pregnant mice (Table 1). These results suggested that the effect of pregnancy on liver regeneration is at least partly attributable to maternally derived factor(s).

In searching for the upstream signals mediating the pregnancy-induced switch, we identified four candidate hormones that are elevated in pregnancy and can activate the Akt/mTORC1 pathway: growth hormone (and its variants), prolactin, leptin and estrogen. Estrogen and growth hormone were shown to increase hepatocyte proliferation after partial hepatectomy (Francavilla et al., 1989; Krupczak-Hollis et al., 2003) and were therefore not tested. We administered prolactin to nonpregnant mice and found no effect on liver regeneration (data not shown).

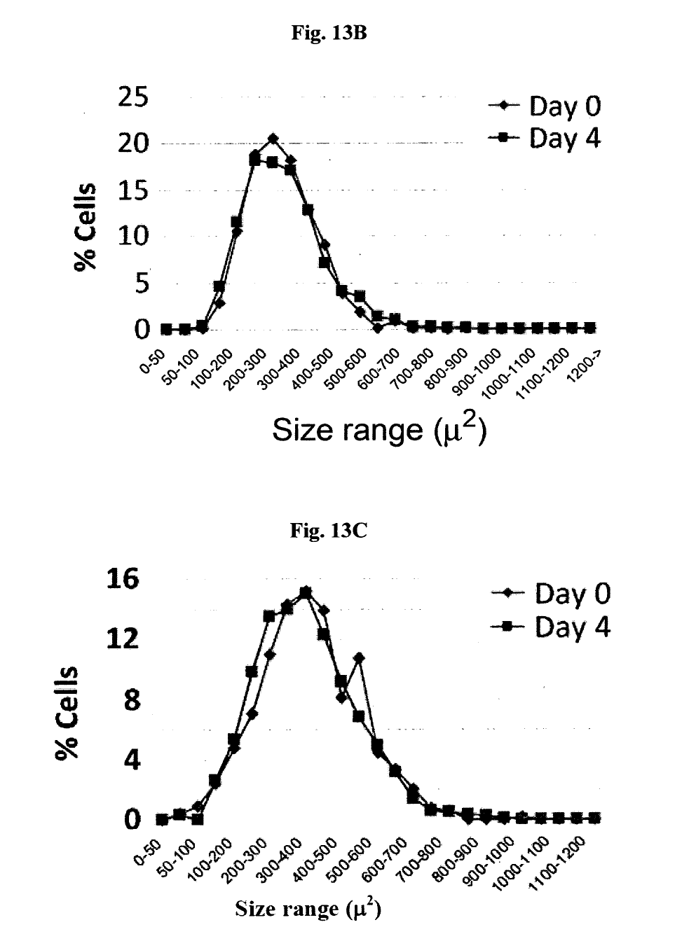

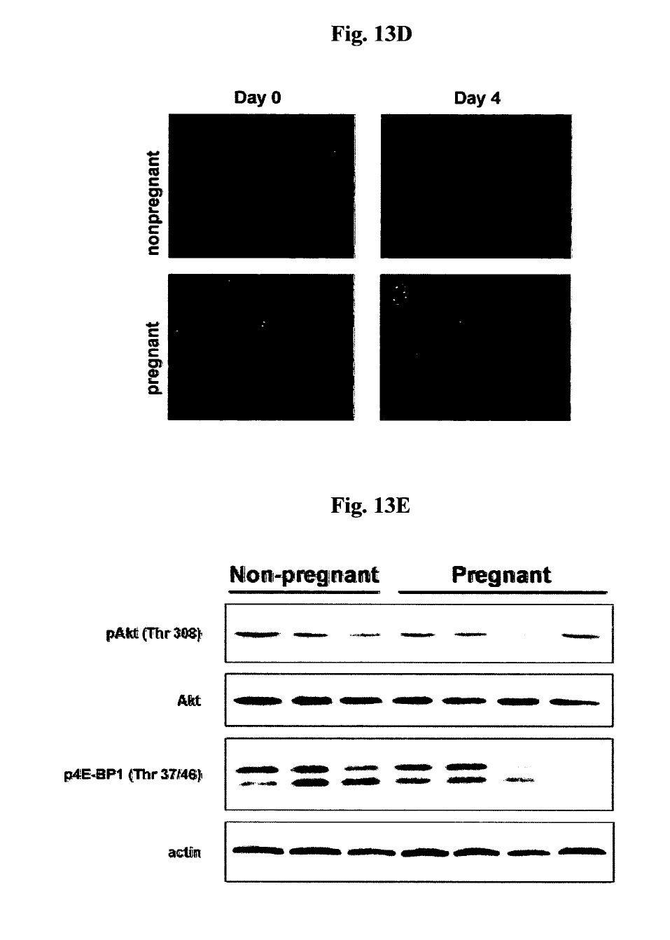

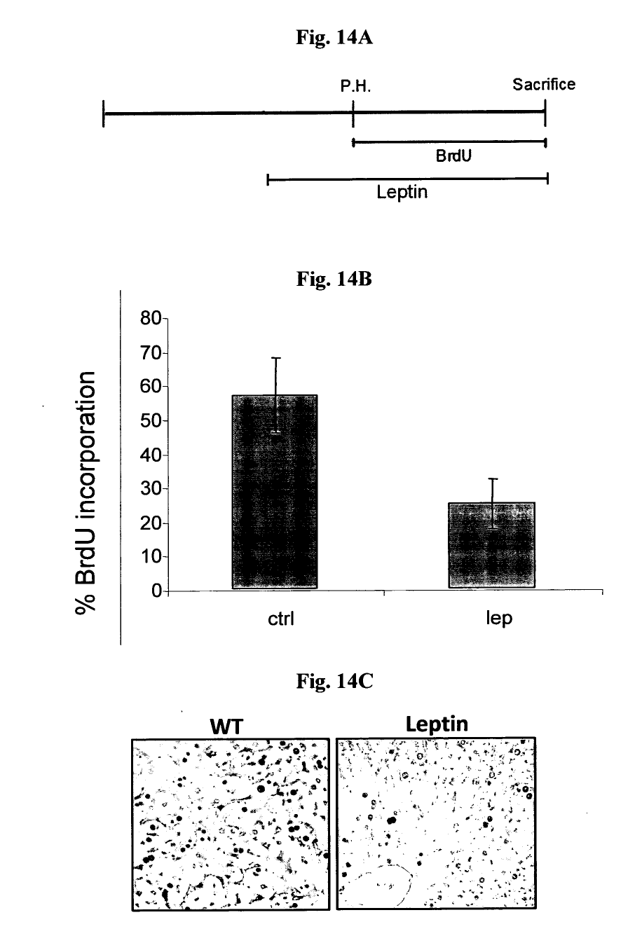

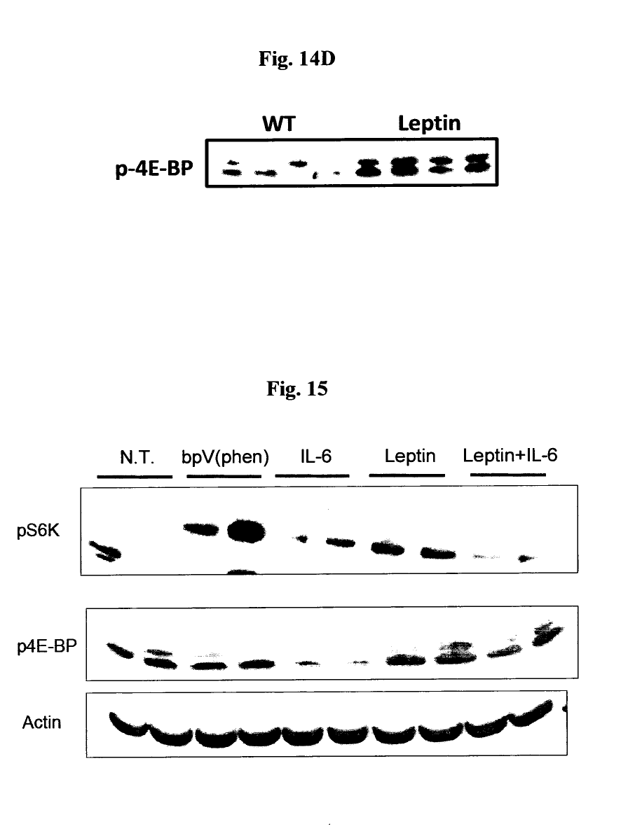

Leptin is an adipokine of the IL6 family and its receptor signals through both the JAK/STAT and the Akt/mTORC1 pathways. Its origin is placental in humans, maternal in mice, and in late pregnancy it is increased by up to 25-fold. To test whether pregnancy-induced leptin accumulation contributes to the proliferation-to-hypertrophy switch in pregnancy we used ob/ob mice, which carry a loss-of-function mutation in leptin. Indeed, hepatocyte proliferation was not decreased in pregnant versus non-pregnant ob/ob mice (FIG. 13A). Moreover, liver regeneration in pregnant ob/ob mice was not accompanied by hepatocyte hypertrophy (FIGS. 13B-D). These findings confirmed that maternal leptin is necessary for the pregnancy-induced module switch. Furthermore, the phosphorylation state of components of the Akt/mTORC1 pathway was not increased in pregnant relative to nonpregnant mice (FIG. 13E FIG. 4e). Two previous studies have shown that leptin attenuates hepatocyte proliferation after partial hepatectomy (one of them in male mice) (Leclercq et al., 2006; Shteyer et al., 2004), consistent with our findings that leptin contributes to the module switch in pregnancy. To test whether leptin can activate the Akt pathway in hepatocytes we administered leptin, IL6, or both to H35 cells (rat hepatoma cell line). bpV(phen) served as control. Cells were harvested and protein extracts were analyzed by Western blot analysis. Indeed, leptin administration resulted in phosphorylation of both S6 kinase and 4E-BP (FIG. 14). To test whether leptin is sufficient to activate the hypertrophy module, we administered leptin or vehicle to nonpregnant female mice prior to and after hepatectomy (FIG. 15A) and assessed liver growth and proliferation. Indeed, leptin induced a marked reduction in hepatocyte proliferation (FIGS. 15B,C) concomitantly with activating the Akt pathway (FIG. 15D).

### REFERENCES

Ben Moshe, T., et al. Role of caspase-8 in hepatocyte response to infection and injury in mice. Hepatology (Baltimore, Md. 45, 1014-1024 (2007).
Conboy I M, Conboy M J, Wagers A J, Girma E R, Weissman I L, Rando T A. 2005. Rejuvenation of aged progenitor cells by exposure to a young systemic environment. Nature 433: 760-764.
Erskine M S. 1998. Pseudopregnancy. In Encyclopedia of reproduction (ed. E Knobil, J D Neill), pp. 120-124. Academic Press, San Diego, Calif.
Francavilla, A., et al. The effect of estrogen and tamoxifen on hepatocyte proliferation in vivo and in vitro. Hepatology (Baltimore, Md. 9, 614-620 (1989).
Haga, S., et al. Compensatory recovery of liver mass by Akt-mediated hepatocellular hypertrophy in liver-specific STAT3-deficient mice. J Hepatol 43, 799-807 (2005).
Haga, S., et al. The survival pathways phosphatidylinositol-3 kinase (PI3-K)/phosphoinositide-dependent protein kinase 1 (PDK1)/Akt modulate liver regeneration through hepatocyte size rather than proliferation. Hepatology (Baltimore, Md. 49, 204-214 (2009).
Hollister, A., Okubara, P., Watson, J. G. & Chaykin, S. Reproduction in mice: liver enlargement in mice during pregnancy and lactation. Life Sci 40, 11-18 (1987).
Inderbitzin, D., et al. Magnetic resonance imaging provides accurate and precise volume determination of the regenerating mouse liver. J Gastrointest Surg 8, 806-811 (2004).
Karnik S K, Chen H, McLean G W, Heit J J, Gu X, Zhang A Y, Fontaine M, Yen M H, Kim S K. 2007. Menin controls growth of pancreatic b-cells in pregnant mice and promotes gestational diabetes mellitus. Science 318: 806-809
Krupczak-Hollis, K., Wang, X., Dennewitz, M. B. & Costa, R. H. Growth hormone stimulates proliferation of old-aged regenerating liver through forkhead box m1b. Hepatology (Baltimore, Md. 38, 1552-1562 (2003). Michalopoulos, G. K. Liver regeneration. J Cell Physiol 213, 286-300 (2007).
Leclercq, I. A., et al. Defective hepatic regeneration after partial hepatectomy in leptin-deficient mice is not rescued by exogenous leptin. Laboratory investigation; a journal of technical methods and pathology 86, 1161-1171 (2006).
Malik, N. M., et al. Leptin requirement for conception, implantation, and gestation in the mouse. Endocrinology 142, 5198-5202 (2001).
Michalopoulos, G. K. & DeFrances, M. C. Liver regeneration. Science 276, 60-66 (1997).
Minamishima, Y. A., Nakayama, K. & Nakayama, K. Recovery of liver mass without proliferation of hepatocytes after partial hepatectomy in Skp2-deficient mice. Cancer Res 62, 995-999 (2002).
Mullany, L. K., et al. Akt-mediated liver growth promotes induction of cyclin E through a novel translational mechanism and a p21-mediated cell cycle arrest. J Biol Chem 282, 21244-21252 (2007).
Nagy, P., et al. Reconstitution of liver mass via cellular hypertrophy in the rat. Hepatology (Baltimore, Md. 33, 339-345 (2001).
Pikarsky, E., et al. NF-kappaB functions as a tumour promoter in inflammation-associated cancer. Nature 431, 461-466 (2004).
Sanders, J. A., Lakhani, A., Phornphutkul, C., Wu, K. Y. & Gruppuso, P. A. The effect of rapamycin on DNA synthesis in multiple tissues from late gestation fetal and postnatal rats. Am J Physiol Cell Physiol 295, C406-413 (2008).
Shiotani, M., Noda, Y. & Mori, T. Embryo-dependent induction of uterine receptivity assessed by an in vitro model of implantation in mice. Biol Reprod 49, 794-801 (1993).
Shteyer, E., Liao, Y., Muglia, L. J., Hruz, P. W. & Rudnick, D. A. Disruption of hepatic adipogenesis is associated with impaired liver regeneration in mice. Hepatology (Baltimore, Md. 40, 1322-1332 (2004).

1. (canceled)

2. (canceled)

3. The method according to claim 15, wherein said liver is damaged due to surgical operation, injury, a disease, a pathological condition, or trauma.

4. The method according to claim 15, wherein the liver is an implanted liver or liver section.

5. The method according to claim 3, wherein said surgical operation includes removal of a tumor.

6. The method according to claim 3, wherein said activator is administered before or after said surgical operation, or both.

7. The method according to claim 3, wherein said disease or pathological condition is selected from the group consisting of: acute liver damage caused by exposure to alcohol; acute viral hepatitis; a metabolic disease resulting in abnormal storage of copper, or iron (hemochromatosis); acute liver damage caused by exposure to drugs or toxins, acute hepatitis caused by autoimmune processes; and acute liver damage caused by obesity or other causes of acute steatohepatitis.

8. The method according to claim 7, wherein said acute liver damage caused by exposure to alcohol is selected from the group consisting of steatosis, alcoholic hepatitis and cirrhosis; said acute viral hepatitis is hepatitis type A; said metabolic disease resulting in abnormal storage of copper is Wilson's disease; and said autoimmune process is autoimmune hepatitis.

9. (canceled)

10. The method according to claim 15, wherein the period of said local or systemic administration is up to 7 days, up to 14 days or up to 30 days.

11. The method according to claim 10, wherein the period of said local or systemic administration is up to 7 days.

12. The method according to claim 15, wherein local administration is achieved by direct application of the activator to the operated liver, or by administration via the portal vein.

13. The method according to claim 15, comprising administering a combination of two or more activators of the AKT/mTOR pathway.

14. (canceled)

15. A method for improving liver regeneration, increasing liver mass or improving liver function, or a combination thereof, in a subject in need of such treatment, the method comprising administering to said subject an activator of the AKT/mTOR pathway.

16. The method of claim 15, wherein said activator is leptin.

17. A method for improving liver regeneration, increasing liver mass or improving liver function, or a combination thereof, in a subject in need of such treatment, the method comprising administering leptin to said subject.

18. The method according to claim 16, wherein said liver is damaged due to surgical operation, injury, a disease, a pathological condition, or trauma.

19. The method according to claim 16, wherein the liver is an implanted liver or liver section.
